(19) United States
(12) Patent Application Publication
(10) Pub. No.: US 2009/0130186 A1
(43) Pub. Date: May 21, 2009

(54) Wound dressing assemblies, systems, and methods formed from hydrophilic polymer sponge structures such as chitosan and incorporating silver nanoparticles

(75) Inventors: Simon J. McCarthy, Portland, OR (US); Jess Kimball, Portland, OR (US)

Correspondence Address: RYAN KROMHOLZ & MANION, S.C., POST OFFICE BOX 26618, MILWAUKEE, WI, 53226, US

(73) Assignee: HemCon Medical Technologies, Inc.

(21) Appl. No.: 12/313,530

(22) Filed: Oct. 21, 2008

Related U.S. Application Data
(63) Continuation-in-part of application No. 10/743,052, filed on Dec. 23, 2003, now Pat. No. 7,371,403.
Application No. 10/743,052 is a continuation-in-part of application No. 10/480,827, filed on Oct. 6, 2004, now Pat. No. 7,482,503.

(51) Int. Cl. A61L 15/16; A61K 33/38; A61K 47/36

(52) U.S. Cl. 424/445; 424/618; 514/777; 977/700; 977/773

(57) ABSTRACT

Silver nanoparticles are mixed with a chitosan solution, to form a chitosan/silver nanoparticle dispersion, which is then subjected to a freeze-drying process, to form a chitosan/silver nanoparticle matrix suitable for use as a wound dressing.

### RELATED APPLICATIONS

This application is a continuation-in-part of co-pending application Ser. No. 10/743,052 filed 23 Dec. 2003, which is a continuation-in-part under 37 C.F.R. § 1.53(h) of U.S. patent application Ser. No. 10/480,827, filed on Dec. 15, 2003, for Wound Dressing and Method of Controlling Severe Life-Threatening Bleeding, which was a national stage filing under 37 C.F.R. § 371 of International Application No. PCT/U502/18757, flied on Jun. 14, 2002.

### FIELD OF THE INVENTION

The invention is generally directed to wound dressings applied on a site of tissue injury, or tissue burns, or tissue trauma, or tissue access to ameliorate bleeding, fluid seepage or weeping, or other forms of fluid loss, as well as provide a protective covering over the site, and to provide antibacterial properties to the site of the tissue injury.

### BACKGROUND OF THE INVENTION

HemCon® Bandages made and sold by HemCon Medical Technologies Inc. (Portland, Oreg.) incorporate a chitosan sponge matrix having superior adhesive properties and resistance to dissolution in high blood flow, which make them well suited for stanching of severe arterial blood flow.

### SUMMARY OF THE INVENTION

The invention provides wound dressing assemblies, systems and methods that utilize nanomaterials such as silver nanoparticles incorporated into hydrophilic polymer sponge structures, such as chitosan.

Other features and advantages of the invention shall be apparent based upon the accompanying description, drawings, and claims.

### BRIEF DESCRIPTION OF THE DRAWINGS

FIG. 1 demonstrates a chitosan powder material being added to a container containing a liquid, preferably deionized water, to form a chitosan solution.

FIG. 2 demonstrates an acid solution being added to the chitosan solution of FIG. 1, with the acid solution being used to further treat the chitosan solution.

FIG. 3A depicts a method of mixing the solution described in FIG. 1, wherein the solution is mixed by manually shaking the container.

FIG. 3B depicts a further method of mixing the solution described in FIG. 1, wherein the solution is mixed by placing the container on a roller apparatus.

FIG. 3C depicts another method of mixing the solution described in FIG. 1, wherein the solution is mixed by using a stirrer in a beaker.

FIG. 4 depicts nanomaterial in the form of silver nanoparticles being added to a liquid, preferably deionized water, to form a silver nanoparticle solution.

FIG. 5 depicts the solution of FIG. 4 being added to the solution of FIG. 2.

FIG. 6 depicts the solution of FIG. 5 being subjected to the rolling apparatus previously depicted in FIG. 3B.

FIGS. 7A and 7B are perspective views of representative molds in which a hydrophilic sponge material desirably comprising chitosan and silver nanoparticles can be formed by freezing and freeze-drying to form the wound dressing assembly shown, respectively, in FIG. 13 and FIG. 15.

FIGS. 8A and 8B are perspective views of a measured volume of chitosan solution being placed into the molds shown in FIGS. 7A and 7B prior to freezing.

FIG. 9 is a perspective view of a freezer in which the chitosan/silver nanoparticle solution, after having been placed into a molds as shown in FIGS. 8A and 8B, is subjected to a prescribed freezing regime and subsequent freeze drying step.

FIG. 10 is a graph showing the phases of a prescribed freezing regime, including a freezing delay interval, that results in the creation of a desirable chitosan/nanomaterial matrix structure.

FIGS. 11A and 11B are perspective views of the removal of a chitosan/nanomaterial matrix structure from the molds shown in FIGS. 8A and 8B after undergoing the freezing regime shown in FIG. 10 as well as a subsequent prescribed freeze-drying process.

FIG. 12 is a perspective view showing the chitosan/nanomaterial matrix structure after removal from the mold, as shown in FIG. 11A.

FIG. 13 is a perspective view of the wound dressing assembly shown in FIG. 12, after having been rolled upon itself for use by a caregiver.

FIGS. 14 and 15 are perspective views of another representative embodiment of a formed hydrophilic sponge material desirably comprising a chitosan/nanomaterial matrix, which is sized and configured as a wound dressing assembly.

FIG. 16 is a perspective view of the wound dressing assembly, shown in roll form in FIG. 13, being unwrapped from the roll form, and then shaped, pushed, and/or stuffed into a wound track by a caregiver.

FIG. 17 is a perspective view of the wound dressing assembly shown in FIG. 12 being cut or torn by a caregiver into smaller segments prior to use.

FIG. 18 is the segment of the wound dressing assembly shown in FIG. 17 by shaped, pushed, and/or stuffed for topical application into a smaller wound track by a caregiver.

FIG. 19 is a perspective view of the wound dressing assembly, shown in FIGS. 14 and 15, being applied to a dressing site by a caregiver.

FIG. 20 depicts three mice being used to analyze the dressing assemblies of the present invention, with one of the mice being subjected to chitosan/silver nanoparticle dressing assembly (A), one being subjected to a chitosan dressing assembly (B), and one of the mice not subjected to any dressing assembly (i.e., the control mouse) (C), to determine antimicrobial effects of the present invention, specifically antimicrobial effects against P. aeruginosa.

FIG. 21 is a graph comparing the results of the effects of the dressing assemblies used on the mice in FIG. 20, showing the percent survival of the mice after a number of days.

FIG. 22 depicts bioluminescence images for each of the mice depicted in FIG. 20, with the images showing the effects of each of the assemblies in FIG. 20 in limiting the spread of P. aeruginosa, compared to the control mouse.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

I. Overview of the Chitosan/Nanomaterial

The present invention provides wound dressing assemblies that incorporate nanomaterials, and, in particular, silver nanoparticles into a chitosan wound dressing matrix.

Generally speaking, silver nanoparticles are mixed with a chitosan solution, to form a chitosan/silver nanoparticle solution, which is then subjected to a freeze-drying process, to form a chitosan/silver nanoparticle matrix suitable for use as a wound dressing. The presence of the silver nanoparticles enhances the antibacterial properties of the matrix.

II. Manufacture of the Chitosan/Silver Nanoparticle Matrix

With reference to FIGS. 1 to 11B, a representative methodology for making the chitosan/silver nanoparticle matrix will now be described. It should be realized, of course, that other methodologies can be used.

1. Preparation of a Chitosan Solution

A portion of the chitosan/silver nanoparticle matrix comprises poly [β-(1→4)-2-amino-2-deoxy-D-glucopyranose, commonly referred to as chitosan. The chitosan selected for the matrix preferably has a weight average molecular weight of at least about 100 kDa, and more preferably, of at least about 150 kDa. Most preferably, the chitosan has a weight average molecular weight of at least about 300 kDa and is derived from chitin obtained from crustacean sources, such as shell fish.

The chitosan used to prepare the chitosan solution preferably has a fractional degree of deacetylation greater than 0.78 but less than 0.97. Most preferably the chitosan has a fractional degree of deacetylation greater than 0.85 but less than 0.95. Preferably the chitosan selected for processing into the matrix has a viscosity at 25° C. in a 1% (w/w) solution of 1% (w/w) acetic acid (AA) with spindle LVI at 30 rpm, which is about 100 centipoise to about 2000 centipoise. More preferably, the chitosan has viscosity at 25° C. in a 1% (w/w) solution of 1% (w/w) acetic acid (AA) with spindle LVI at 30 rpm, which is about 125 centipoise to about 1000 centipoise. Most preferably, the chitosan has viscosity at 25° C. in a 1% (w/w) solution of 1% (w/w) acetic acid (AA) with spindle LV1 at 30 rpm, which is about 400 centipoise to about 800 centipoise.

As depicted in FIG. 1, a chitosan solution is preferably prepared at 25° C. by addition of water 12 to solid chitosan flake or powder 14 within a container. The solid chitosan flake 14 is dispersed in the liquid by agitation, stirring or shaking.

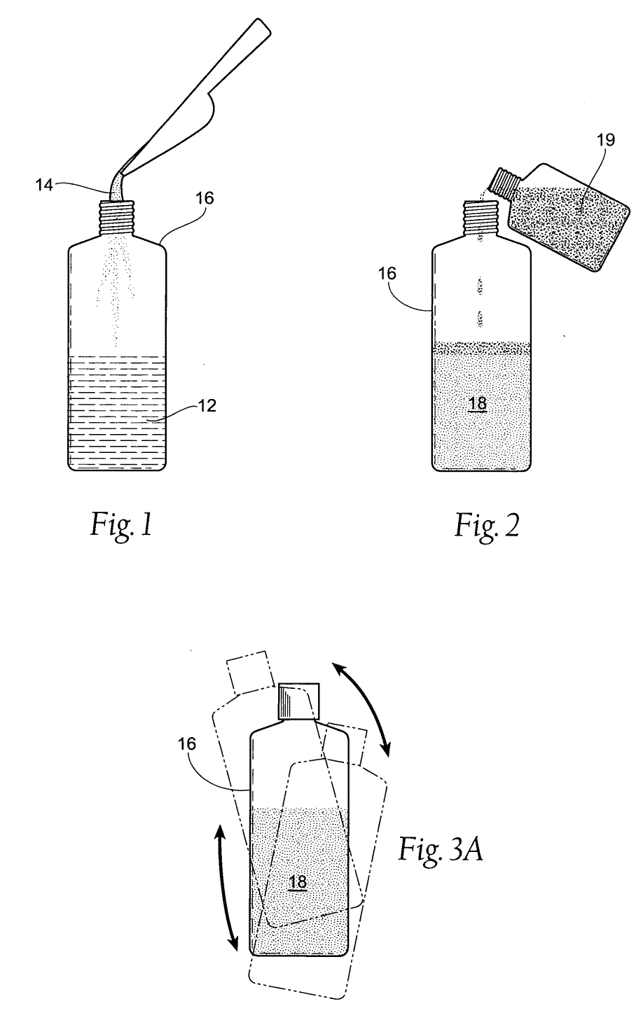

The chitosan/water solution 18 is further processed by the addition of an acid to the chitosan/water solution, as depicted in FIG. 2. The chitosan/water is desirably placed into solution with an acid 20, such as glutamic acid, lactic acid, formic acid, hydrochloric acid, glycolic acid, and/or acetic acid. Among these, hydrochloric acid and acetic acid are most preferred.

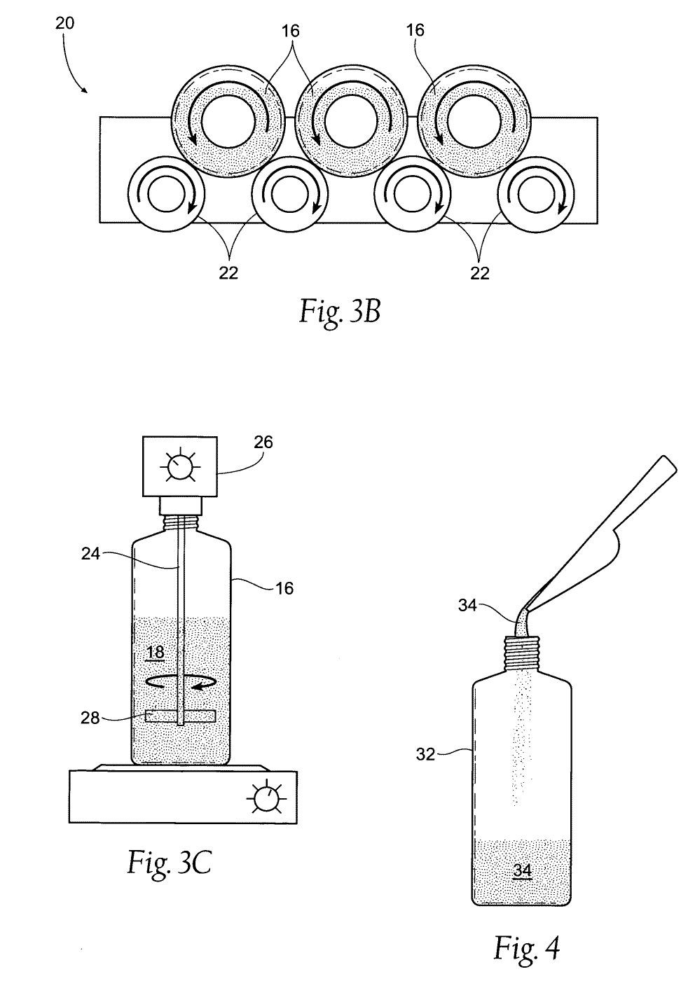

The acid component is added and mixed through the dispersion to cause dissolution of the chitosan solid. The rate of dissolution will depend on the temperature of the solution, the molecular weight of the chitosan and the level of agitation. Preferably the dissolution step is performed within a closed tank reactor with agitating blades or a closed rotating vessel (see FIGS. 3B and 3C). This ensures homogeneous dissolution of the chitosan and no opportunity for high viscosity residue to be trapped on the side of the vessel. Preferably the chitosan solution percentage (w/w) is greater than 0.5% chitosan and less than 2.7% chitosan. More preferably the chitosan solution percentage (w/w) is greater than 1% chitosan and less than 2.3% chitosan. Most preferably the chitosan solution percentage is greater than 1.5% chitosan and less than 2.1% chitosan. Preferably the acid used is acetic acid. Preferably the acetic acid is added to the solution to provide for an acetic acid solution percentage (w/w) at more than 0.8% and less than 4%. More preferably the acetic acid is added to the solution to provide for an acetic acid solution percentage (w/w) at more than 1.5% (w/w) and less than 2.5%.

As noted, FIGS. 3A-3C present various methods of mixing the water and chitosan to form the chitosan solution, as well as various methods of mixing the water/chitosan solution with an acid. FIG. 3A demonstrates manual, physical agitation of the water and chitosan. After the chitosan and water are added to one another as shown in the container shown in FIG. 1, the container will be grasped and shaken, either by a machine or by a person, until the chitosan is sufficiently wetted. FIG. 3B depicts a rolling apparatus 20 that will also allow the chitosan powder to be sufficiently wetted or mixed with the water. The rolling apparatus 20 contains a plurality of rollers 22. Each of the containers 16 is placed on the rollers 22, with the rollers rotating the containers 16. The rolling apparatus may also be used to combine with chitosan/water solution with the acid, as discussed with respect to FIG. 2.

FIG. 3C shows yet another method of mixing the chitosan and water. A rotor 24 attached to a motor 26 is inserted into the container 16, or another container, such as a beaker. The rotor 20 and motor 22 are standard as used and understood in the art, with the paddles 28 of the rotor 20 preferably being positioned in the bottom third of the beaker 12, to thoroughly mix the chitosan and solution. Provided that the chitosan material is thoroughly wetted and further sufficiently mixed with the acid material, the mixing method would be acceptable according to the present invention.

2. Preparation of the Nanomaterial Solution

In the illustrated embodiment, the nanomaterial comprises silver nanoparticle material comprising a nanocrystalline silver material, such as SmartSilver, manufactured by NanoHorizons, Inc., State College, Pa., or material received from NanoSense, located in Galway, Ireland. This silver nanoparticle material normally is supplied in dry, flake form. The silver nanoparticle material consists of metallic silver nanoparticles that are combined with a polymer stabilizer, but other nanomaterials, such as silver salt compounds, could be utilized.

The silver nanoparticle material 30 is added to a container 32 of deionized water 34, as shown in FIG. 4. The container 34 is placed on a rolling apparatus 20, as shown in FIG. 3B to form a silver nanoparticle dispersion, which will also be referred to as a nanosilver dispersion. It is also possible that the devices and methods described in FIGS. 3A and 3C could be employed to mix the silver nanoparticle material and the water, as well.

3. Forming the Chitosan/Nanomaterial Solution

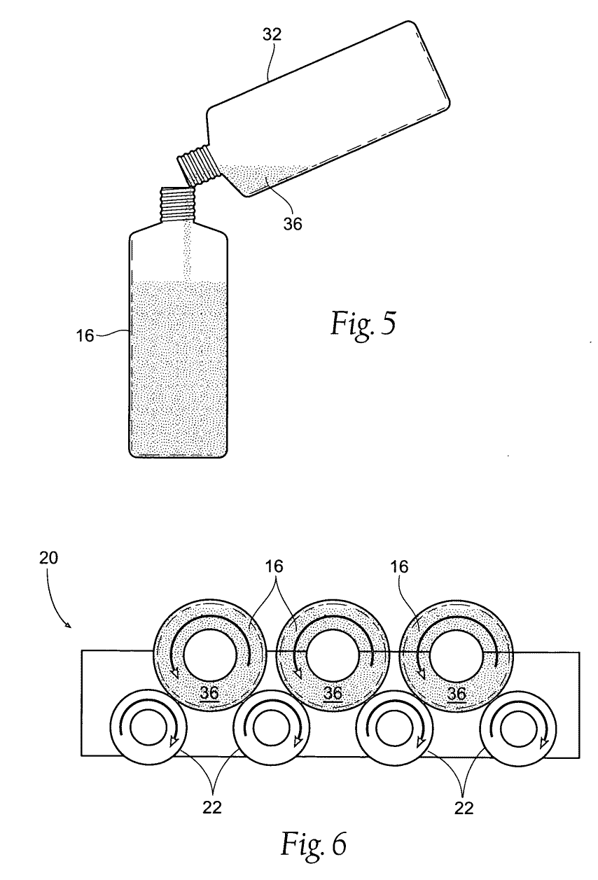

As depicted in FIG. 5, the nanosilver dispersion from FIG. 4 is added to the chitosan solution from FIG. 2 and subjected to the rolling apparatus 20 to form a homogenous mixture 36 (FIG. 6). Citric acid and glycerol can also be added to the chitosan/nanosilver dispersion, either before or after the addition of the nanosilver solution, and thoroughly mixed to form a homogenous chitosan/nanosilver dispersion. The homogenous solution will then be subjected to a freeze-drying process. It should be noted that the nanoparticles are not soluble in solution, but are mixed to from a homogenous dispersion/suspension within the chitosan solution.

It is possible that the nanosilver material can be added directly to the chitosan solution, without first making it into a mixture. However, adding the dry material directly to the chitosan solution can form insoluble precipitates, which are not necessarily beneficial and can effect measuring the final concentration of silver within the final dressing assembly.

4. Degassing the Aqueous Chitosan/Nanomaterial Chitosan Solution

If desired, the chitosan/nanosilver biomaterial can be degassed of general atmospheric gases. Degassing can remove sufficient residual gas from the chitosan/nanosilver biomaterial so that, on undergoing a subsequent freezing operation, the gas does not escape and form unwanted large voids or large trapped gas bubbles in the subject wound dressing product. The degassing step may be performed by heating a chitosan/nanosilver biomaterial, typically in the form of a chitosan solution having a nanosilver material evenly suspended or dispersed through the chitosan solution, and then applying a vacuum thereto. For example, degassing can be performed by heating a chitosan/nanosilver solution to about 45° C. immediately prior to applying vacuum at about 500 mTorr for about 5 minutes while agitating the solution.

In one embodiment, certain gases can be added back into the solution to controlled partial pressures after initial degassing. Such gases would include but are not limited to argon, nitrogen and helium. An advantage of this step is that solutions containing partial pressures of these gases form micro-voids on freezing. The microvoid is then carried through the sponge as the ice-front advances. This leaves a well defined and controlled channel that aids sponge pore interconnectivity.

5. Freezing the Aqueous Chitosan/Nanosilver Solution

The form producing steps for the chitosan/nanomaterial matrix are typically carried out from the chitosan/nanomaterial dispersion. The form producing steps can he accomplished employing techniques such as freezing (to cause phase separation), non-solvent die extrusion (to produce a filament), electro-spinning (to produce a filament), phase inversion and precipitation with a non-solvent (as is typically used to produce dialysis and filter membranes) or solution coating onto a preformed sponge-like or woven product.

In the illustrated embodiment, the chitosan/nanomaterial biomaterial—now in acid solution, thoroughly mixed and optionally degassed, as described above—is subjected to a form producing step that includes a controlled freezing process. The controlled freezing process is carried out by cooling the chitosan/nanomaterial biomaterial mixture within a mold 122 or 122′.

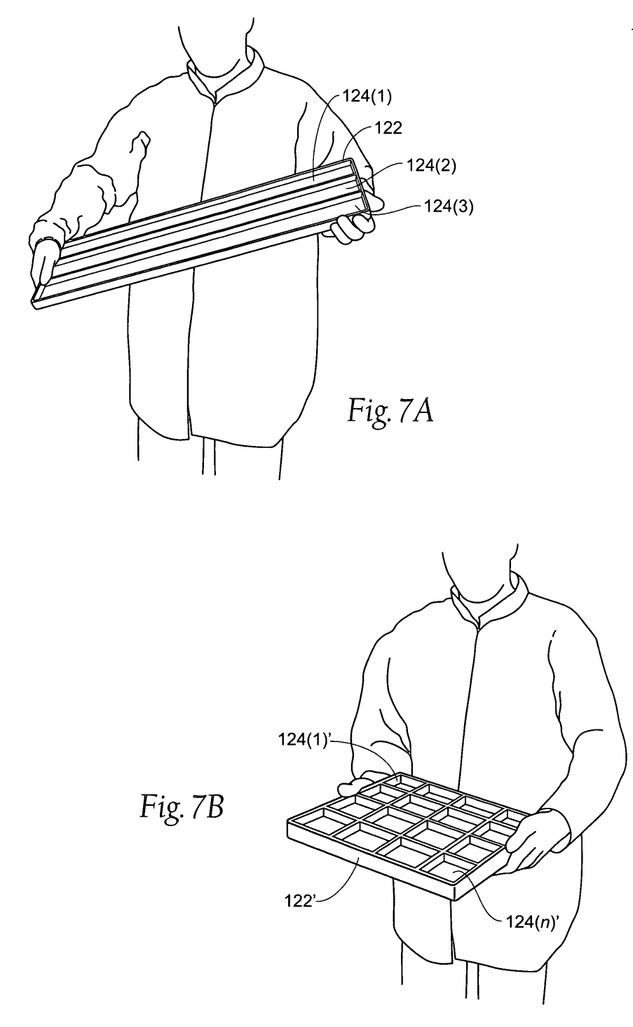

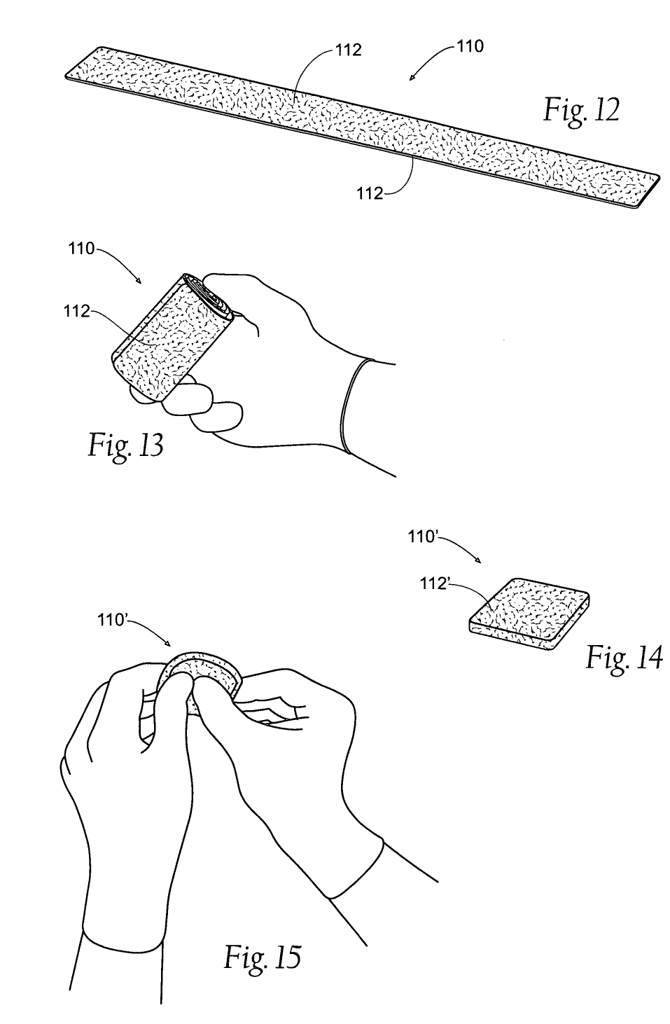

The mold 122 or 122′ can be variously constructed. As shown in FIGS. 7A and 7B, the mold 122 and 122′ for forming the chitosan/nanomaterial matrix 112 or 112′ (FIGS. 12 and 14, respectively) can be made from a metallic material, e.g., Mic 6 aluminum, although other metallic materials and alloys can be used, such as iron, nickel, silver, copper, titanium, titanium alloy, vanadium, molybdenum, gold, rhodium, palladium, platinum and/or combinations thereof.

In a representative embodiment for creating a chitosan/nanomaterial matrix 112 like that shown in FIG. 12, the mold 122 measures overall 30 inches by 9.8 inches, and is compartmentalized into three mold chambers 124(1), 124(2), and 124(3), each 3 inches in width and 0.051 inch in depth. The mold chambers 124(1), 124(2), and 124(3) are desirably coated with a thin, permanently-bound, fluorinated release coating formed from polytetrafluoroethylene (Teflon), fluorinated ethylene polymer (FEP), or other fluorinated polymeric materials.

As FIG. 7B shows, the mold 122′ for forming the smaller matrix 112′ (FIGS. 14 and 15) can be made from a plastic material compartmentalized into multiple small wells or chambers 124(1)′ to 124(n)′ for forming multiples of assemblies 112′ at one time.

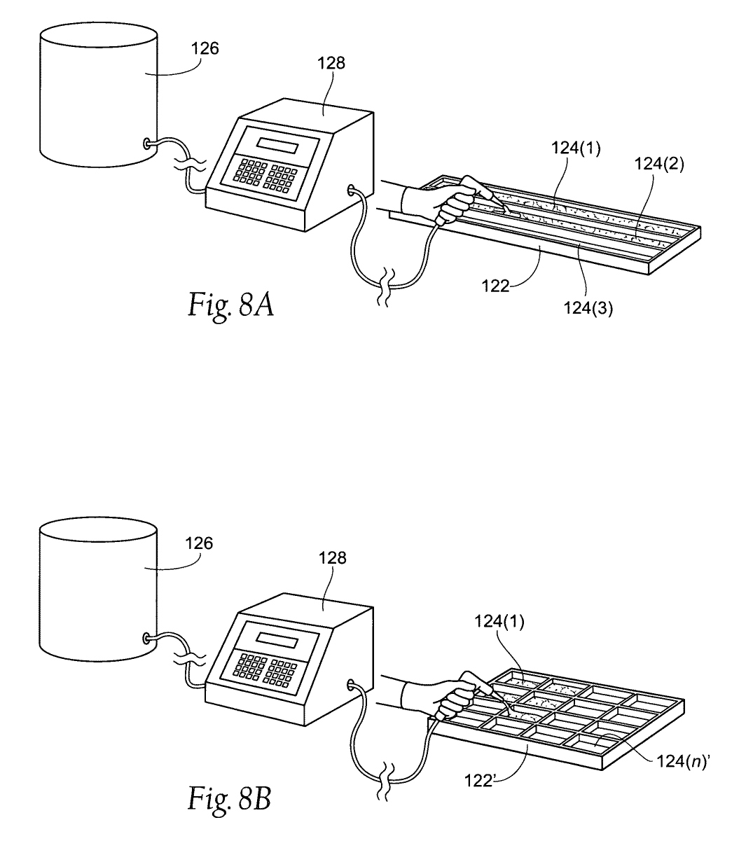

As FIGS. 8A and 8B show, a preselected volume of the chitosan/nanomaterial biomaterial dispersion is conveyed from a source 126 into each mold chamber 124(1), 124(2), and 124(3) or 124(1)′ to 124(n)′ using, e.g., a positive displacement pump 128. Given the mold dimensions disclosed above for creating the chitosan/nanomaterial matrix 112 (FIG. 8A), in a representative embodiment, 450 gr +/−13 of chitosan/nanomaterial biomaterial dispersion is conveyed into each mold chamber 124(1), 124(2), and 124(3): Adding a lesser volume of the chitosan/nanomaterial biomaterial dispersion will result in a matrix that, after molding, possesses a thinner cross section and therefore an ultimately thinner finished chitosan/nanomaterial matrix.

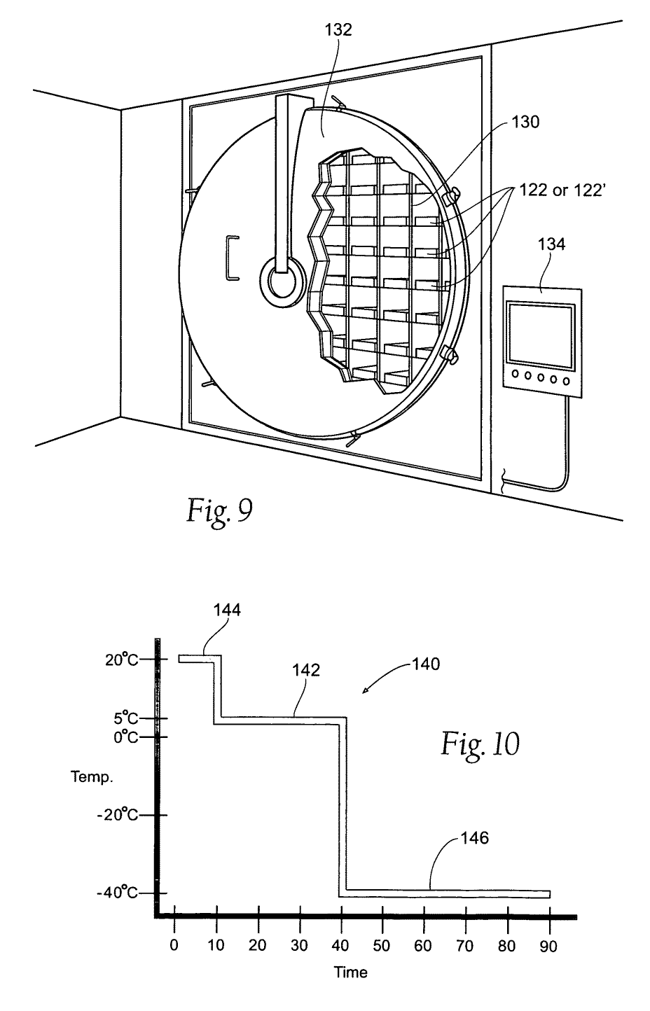

The mold 122 or 122′ and chitosan/nanomaterial biomaterial dispersion are then located on flat stainless-steel heating/cooling shelves 130 within a freeze dryer 132 (FIG. 9). The flat base of each mold chamber 124(1), 124(2), and 124(3) or 124(1)′ to 124(n)′ is placed in close thermal contact with the flat stainless-steel heating/cooling surface of the shelf 130. A microprocessor controller 134 carries out the prescribed steps of the freezing process control algorithm.

Within the freezer 132, under the control of the controller 134, the temperature of the chitosan/nanomaterial biomaterial dispersion is ultimately lowered from room temperature (e.g., about 20° C.) to a final temperature well below the freezing point (e.g., minus 40° C.). The chitosan/nanomaterial biomaterial dispersion within each mold chamber 124(1), 124 (2), and 124 (3) or 124(1)′ to 124(n)′ loses heat uniformly through the shelf cooling surface and freezes. In this process, the chitosan/nanomaterial biomaterial dispersion undergoes phase separation, which begins to form the desired structure of the matrix.

As shown in FIG. 10, a representative freezing regime 140 implemented by the controller 134 includes lowering the chitosan/nanomaterial biomaterial dispersion temperature from room temperature to a final temperature below the freezing point, and includes at least one intermediate delay interval 42 that holds a temperature condition for a prescribed period of time at a prescribed increment above the freezing point. In the illustrated embodiment, the freezing regime 140 includes a first interval 144 that maintains a desired start temperature at or near room temperature (e.g., 20° C.) for a prescribed period of time (e.g., 10 minutes). The freezing regime 140 next drops the temperature to an intermediate temperature, which is held during the delay interval 142. The intermediate temperature is desirably between 2° C. and 10° C. The delay interval 142 is desirably between 20 minutes and 40 minutes. In a representative embodiment, the intermediate temperature is 5° C. and the delay interval 142 is 30 minutes. The freezing regime 140 includes a final interval 146 that lowers the temperature from the intermediate temperature to the desired final temperature, which is maintained for a prescribed period. In a representative embodiment, the final temperature is minus 40° C., and the prescribed period of time is 50 minutes.

4. Freeze Drying the Chitosan/Nanomaterial/Ice Matrix

The frozen chitosan/nanomaterial/ice matrix desirably undergoes water removal from within the interstices of the frozen material. This water removal step may he achieved without damaging the structural integrity of the frozen chitosan/nanomaterial biomaterial. This may be achieved without producing a liquid phase, which can disrupt the structural arrangement of the ultimate chitosan/nanomaterial matrix 112 and 112′. Thus, the ice in the frozen chitosan/nanomaterial biomaterial passes from a solid frozen phase into a gas phase (sublimation) without the formation of an intermediate liquid phase. The sublimated gas is trapped as ice in an evacuated-condenser chamber at substantially lower temperature than the frozen chitosan/nanomaterial biomaterial. Since the spherulitically nucleated structures that are desirably present within the matrix 112 and 112′ often retain considerable moisture due to an impermeable shell structure that forms around the ice core, conditions must be maintained during the water removal step to keep the matrix temperature below its collapse temperature, i.e., the temperature at which the ice core within the structure could melt before it is sublimated.

The preferred manner of implementing the water removal step is by freeze-drying, or lyophilization within the freezer 132. Freeze-drying of the frozen chitosan/nanomaterial biomaterial can be conducted by further cooling the frozen chitosan/nanomaterial biomaterial. Typically, a vacuum is then applied. Next, the evacuated frozen chitosan/nanomaterial material is subject to ramped heating and/or cooling phases in the continued presence of a vacuum.

In a representative embodiment, following the freezing regime 140, freeze drying conditions are maintained for removing water without collapse of the matrix 112 and 112′. In a representative embodiment, for example, a prescribed freeze drying temperature, e.g., minus 50° C. is maintained for a preferred time period (e.g., between 1 and 3 hours), while a vacuum, e.g., in the amount of about 170 mTorr, is applied during this time.

Further freeze drying at higher temperatures may be conducted during subsequent drying phases, while maintaining vacuum pressure. The times and temperatures of the drying phase can change depending upon fill volume, mold configuration, lyophilizer capabilities, etc. Step changes are made to keep the matrix temperature below its collapse temperature. The temperature of the matrix 112 and 112′ is kept as high as possible during the drying phases, but still below the collapse temperature, to provide the shortest cycle time possible. The shelf temperature is ramped up and then down again because high rates of initial sublimation cools the matrix temperature, and as sublimation wanes, matrix temperature increases.

Further details of the freezing and freeze-drying process are disclosed in co-pending U.S. patent application Ser. No. 11/900,854, filed Sep. 23, 2007, which is incorporated herein by reference.

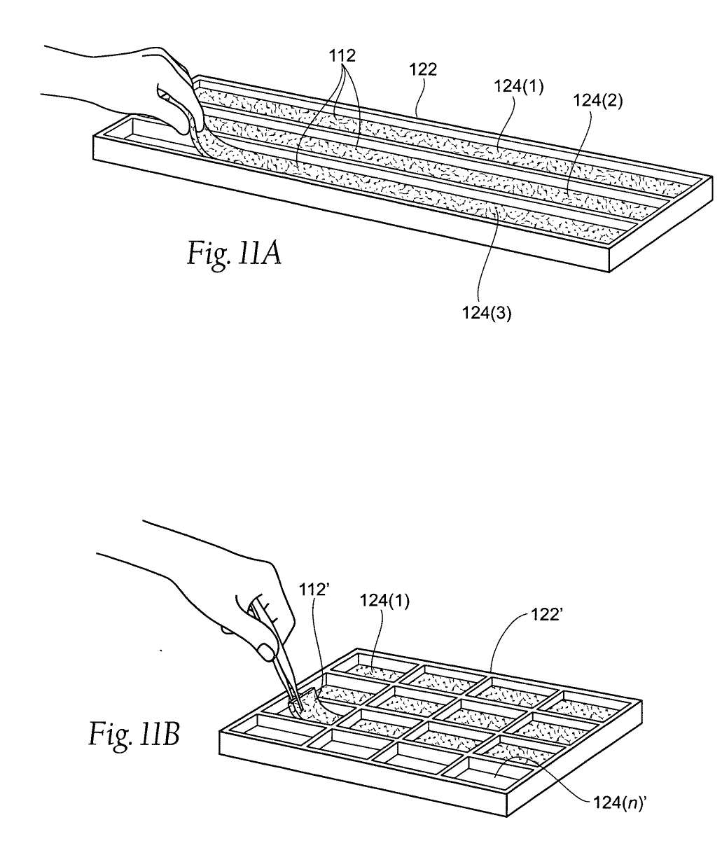

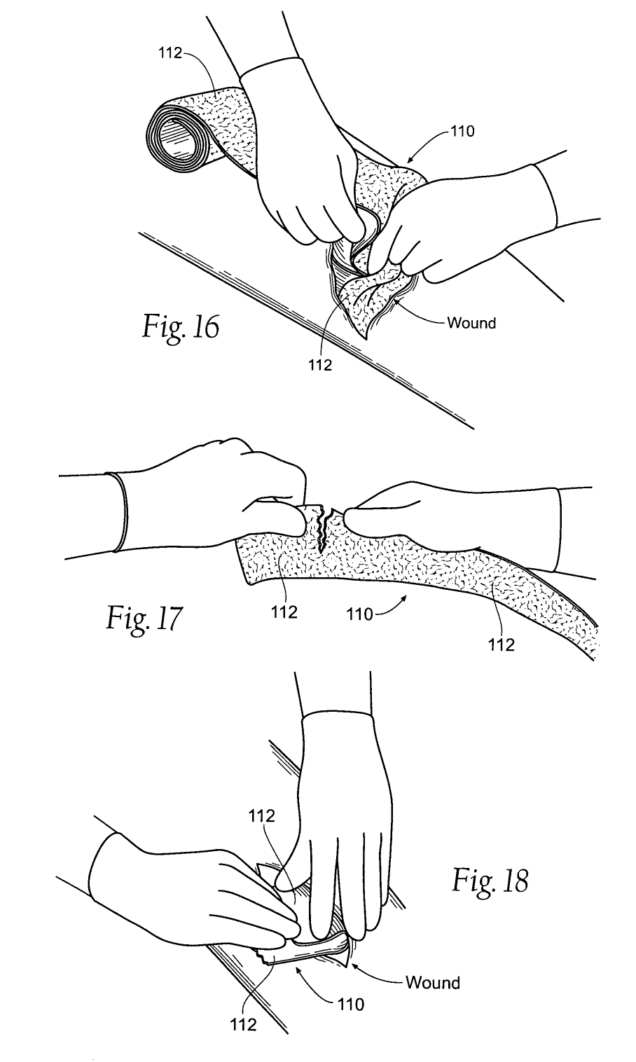

As shown in FIGS. 11A and 11B, the formed, freeze dried matrix 112 and 112′ can be removed from the mold chamber 124(1), 124(2), and 124(3) and 124(1)′ to 124(n)′. When removed from the mold chamber 124(1), 124(2), and 124(3) (see FIG. 15), the formed, freeze-dried matrix 112 measures 28 inches by 2.75 inches, with a thickness of about 0.23 to 0.28 inches. When removed from the mold 122 (see FIG. 16), the formed matrix 112 exhibits inherently suppleness, i.e., it possesses the inherent flexibility and lack of brittleness and stiffness as described above. When removed from the mold chambers 124(1)′ to 124(n) (see FIG. 11B), the smaller formed freeze-dried matrix 112′ also possesses the same inherent suppleness, as shown in FIG. 15.

When removed from the mold chamber, the chitosan/nanomaterial matrix 112 and 112′ has a density at or near about 0.03 g/cm³ as a result of the freezing regime 40. For purposes of description, this structure will be called an “uncompressed chitosan/nanomaterial matrix.”

5. Subsequent Processing of the Chitosan Matrix

If desired, either matrix 112 and 112′ can be subject to further processing to impart other physical characteristics and otherwise optimize the matrix 112 and 112′ for its intended end use.

For low bleeding hemostasis and/or targeted antibacterial/antiviral wound dressing situations, and/or for dental indications, further processing may not be warranted, because the supple uncompressed matrix 112′ (shown ready for use in FIGS. 14 and 15) has, after freezing and freeze-drying as described above, the requisite adhesion strength, cohesion strength, dissolution resistance, flexure, and conformity to perform well in such environments. The uncompressed dry matrix 112′ can be removed from the mold, pouched, and sterilized (as will be described later) without subsequent matrix processing steps.

However, subsequent processing of the matrix may desired after drying and prior to packing and sterilization, for example, when the wound dressing assembly 110 is intended to be, in use, exposed to higher volume blood flow or diffuse bleeding situations, or when exposure to relatively high volume of fluids is otherwise anticipated, as shown in FIG. 16.

Representative subsequent matrix processing steps can include, e.g., densification by heat and pressure to increase the density of the uncompressed dry chitosan/nanomaterial matrix to a density greater than or equal to 0.1 g/cm³, desirably between 0.1 g/cm³ and about 0.5 g/cm³, and most desirably about 0.2 g/cm³. For example, the uncompressed chitosan/nanomaterial matrix can be placed between heated platens, including one or more spacers of defined dimensions to ensure consistent thickness. The compression temperature is preferably not less than about 60° C., more preferably it is not less than about 75° C. and not more than about 85° C. The compression load of the heated platens reduces the thickness of the uncompressed chitosan/nanomaterial matrix from about 0.23 to 0.28 inches to about 0.036 inch (i.e., about 0.9 mm), thereby increasing the density of the matrix from about 0.03 g/cm³ to a target density of, e.g., about 0.2 g/cm³. FIG. 12 shows a chitosan/nanomaterial matrix, following densification.

Other representative subsequent matrix processing steps can include, e.g., mechanical softening. The softening can be accomplished, e.g., by the mechanical manipulation of the matrix between an array of upper and lower rollers, which knead the matrix, thereby mechanically softening it. As shown in FIG. 13, the elongated tissue dressing matrix 112 shown in FIG. 12 can, after softening, be manually rolled tightly upon itself, to form a roll that can be as small as about 1.5 inches (38 mm) in diameter, depending upon how tightly rolled the matrix is.

Other representative subsequent matrix processing steps can include, e.g., preconditioned by heating in an oven at a temperature of preferably up to about 75° C., more preferably to a temperature of up to about 80° C., and most preferably to a temperature of preferably up to about 85° C. Preconditioning by heating can typically be conducted for a period of time up to about 0.25 hours, preferably up to about 0.35 hours, more preferably up to about 0.45 hours, and most preferably up to about 0.50 hours.

Further details of the subsequent matrix processing steps are disclosed in co-pending U.S. patent application Ser. No. 11/900,854, filed Sep. 23, 2007, which is incorporated herein by reference.

It may be desirable, to apply a backing to the chitosan/nanomaterial matrix. The backing isolates a caregiver's fingers and hand from the fluid-reactive chitosan/nanomaterial matrix.

Before use, the wound dressing assembly 110 is desirably vacuum packaged in an air-tight heat sealed foil-lined pouch. The wound dressing assembly 110 can be subsequently terminally sterilized within the pouch by use of gamma irradiation.

It should be appreciated that other nanomaterials, such as nanofibers, can be incorporated into a freeze-dried chitosan matrix. Nanofibers generally are defined as fibers with diameters less than 100 nanometers. They can be produced by conventional interfacial polymerization and electrospinning. Nanofibers can be chopped into small particles and suspended in or dispersed into a chitosan solution (in the same manner as the silver nanoparticles), which is then freeze-dried into a wound dressing matrix. The presence of the nanofibers increase the surface area and strength of the wound dressing matrix. As another example, chitosan can itself be electrospun into nanofiber form, then reacetylated into chitin, and dispersed in solution with chitosan (in the same manner as the silver nanoparticles), which is then freeze-dried into a wound dressing matrix.

III. Uses for the Chitosan/Silver Nanoparticle Matrix

The wound dressing assemblies comprising silver nanoparticles incorporated into a freeze-dried chitosan matrix can be used, e.g., (i) to stanch, seal, or stabilize a site of tissue injury, tissue burn, tissue trauma, or tissue access; or (ii) to form an anti-microbial barrier; or (iii) to form an antiviral patch; or (iv) to intervene in a bleeding disorder; or (v) to release a therapeutic agent; or (vi) to treat a mucosal surface; or (vii) to dress a staph or MRSA infection site; or (viii) in various dental surgical procedures, or (ix) combinations thereof.

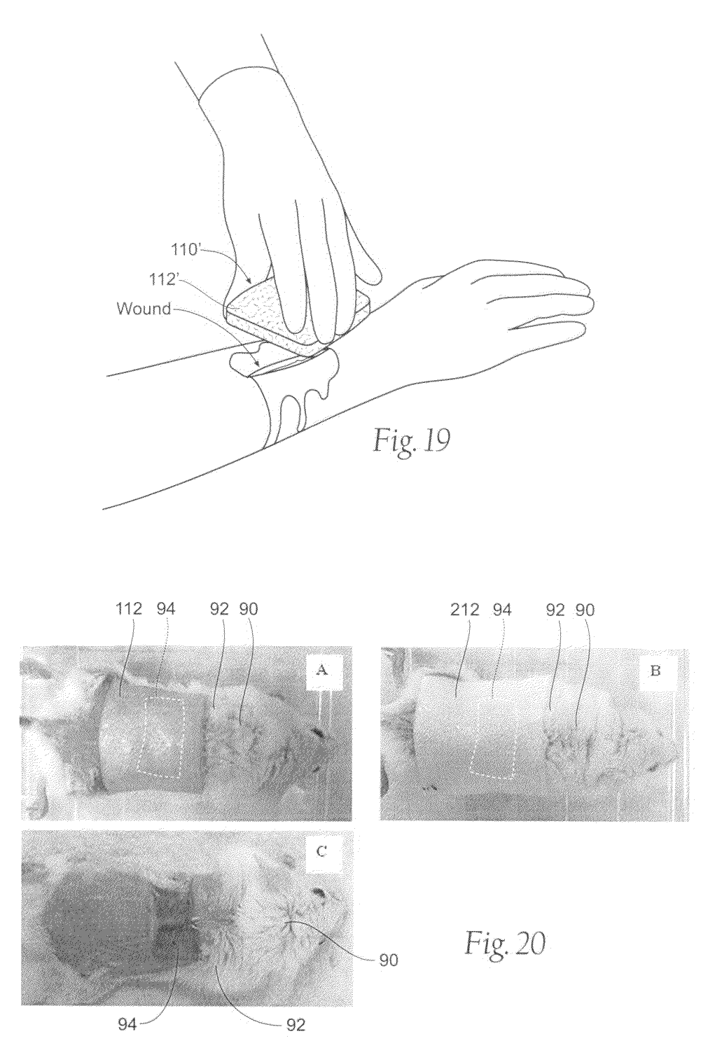

The wound dressing assembly 110 can be readily sized and configured to be shaped, pushed, and/or stuffed into a wound track, as FIG. 16 shows. The wound dressing matrix 12 can be readily cut or torn into smaller segments (see FIG. 17) for topical application upon or insertion within a smaller wound (see FIG. 18). For a smaller wound (as FIG. 18 shows), once torn or cut into a smaller segment, the segment of the dry wound dressing matrix 112 can be readily folded into a “C” shape or another configuration to facilitate its insertion into a wound track. As shown in FIG. 19, the wound dressing assembly 110′ can be sized and configured with smaller, preformed dimensions for topical application for, e.g., low bleeding hemostasis and/or antibacterial/antiviral wound dressing applications.

Table 1 lists various different chitosan/nanosilver compositions that can be prepared according to the invention. Each of the groups was prepared with various combinations of acids and/or glycerol.

TABLE 1
Chitosan/Silver Prototypes

| | A | B | C | D | Range |
|---|---|---|---|---|---|
| Chitosan | X | X | X | X | 1-2% |
| Acetic Acid | X | X | X | X | 0.42-2% |
| Lactic Acid | | | X | X | 0-0.65% |
| Citric Acid | | X | X | X | 0-0.8% |
| Glycerol | | | | X | 0-0.5% |
| Silver | X | X | X | X | 0-1.0% |
| Nanoparticles | | | | | |
| Densified | Yes | No | No | No | |
| Precondition | Yes | No | No | No | |
| by Heat | | | | | |

Group A was processed further to form a compressed matrix and further preconditioned by heating (as described above), while Groups B to D were not subjected to further processing after freeze-drying. While all of the groups demonstrated desirable wound healing characteristics in terms of antibacterial activity, resistance to dissolution, adhesion, and absorbency—Group D demonstrated the most favorable results. In Group D, the lactic acid and the acetic acid were used to dissolve the chitosan, and the citric acid was used as an ionic cross-linking agent to provide resistance for the final matrix composition of dissolving in fluids. Group D matrices were shown to absorb ≧15 times their weight in water. Visual observation of the matrices when exposed to fluid indicates that the glycerol content of the matrix may influence swelling, with higher glycerol levels associated with greater water absorption and swelling.

### Example 1

### Comparison of Antimicrobal Effects

Dressing assemblies according to the present invention were tested to analyze the antimicrobial effects of the dressing assemblies. Specifically, the dressing assemblies were applied to mice that were subjected to the bacteria, P. aeruginosa ATCC 19660. The procedure and results are discussed below and with respect to FIGS. 20-22.

A. Test Animals Used (Mice)

Adult female BALB/c mice (Charles River, Wilmington, Mass.), 6-8 week old and weighing 17-21 g, were used in the study. The mice were housed one per cage and maintained on a 12-hour light/dark cycle with access to food and water ad libitum. All animal procedures were approved by the Subcommittee on Research Animal Care of Massachusetts General Hospital and met the guidelines of National Institutes of Health.

B. Bacteria Strain Tested P. aeruginosa ATCC 19660, which causes septicemia after intraperitoneal injection and has been shown to be invasive in mice with skin burns, was employed in the study. The stable bioluminescent variants of this strain carried the entire bacterial lux operon integrated in their chromosomes for stable luciferase expression that allowed them to be used for bioluminescent imaging (Xenogen Inc). Bacteria were grown in a brain-heart infusion (BHI) medium in an orbital incubator (37° C.; 100 rpm) to an optical density of 0.6 at 650 nm that corresponds to 10⁸ cells/mL (mid-log phase). This suspension was centrifuged, washed with phosphate buffered saline (PBS), and re-suspended in PBS at the same density. Luminescence was routinely measured on 100-uL aliquots of bacterial suspensions in 96-well black-sided plates, by use of a Victor-2 1420 Multilabel Plate Reader (EG&G Wallac).

C. Preparation of the Mice

As shown in FIG. 20, the mice 90 were shaved on the back 92 and depilated with Nair® (Carter-Wallace Inc) hair depilatory. The next day mice were anesthetized with intraperitoneal injections of ketamine/xylazine solution, and burns 94 were created by applying two pre-heated (92-95° C.) brass blocks (10-mm×10-mm; Small Parts, Inc., Miami, Fla.) to the opposing sides of an elevated skin-fold on the dorsal surface of the mouse for 60 seconds, which correlates to non-lethal, full-thickness, third-degree burns. The combined brass block area was 20 mm×10 mm giving an area of 200 mm², corresponding to a 5% of total body surface area (TBSA). Immediately after the creation of burn the mice were resuscitated with intraperitoneal injections of 0.5 mL sterile saline (Phoenix Scientific Inc).

Five minutes after the creation of burn (to allow the burn to cool down), a suspension (40-μL) of bacteria in sterile PBS containing 10⁸ cells was inoculated onto the surface of each burn with a yellow-tipped pipette and then was smeared onto the burn surface with an inoculating loop.

D. Treatment of the Mice

As shown in FIG. 20, dressing assemblies according to the present invention were applied to the infected burns 15 minutes after the application of bacteria, allowing the bacteria sufficient time to bind to the burned tissue. One of the dressing assemblies 212 comprised a chitosan/silver nanomaterial (FIG. 20 (A)), one dressing assembly 112 included a chitosan material (FIG. 20(B)), while the third mouse did not have any dressing assembly applied (FIG. 20(C)), which was considered the control mouse. It should be noted that the dressing assembly 212 was prepared in the same fashion described above as the dressing assembly 112 containing the silver nanomaterial, except that the silver nanomaterial was not added to the prepared matrix. That is, the steps of forming the chitosan solution, degassing, freeze-drying, etc., are the same for both the chitosan assembly and the chitosan/silver assembly.

To adhere the dressing assemblies to the burns, both dressing assemblies 112, 212 were moistened with MilliQ water before application. In contrast to human third degree burns, mouse third degree burns have a dry texture, irrespective of whether they have been contaminated or infected with bacteria. It was therefore necessary to regularly moisten both dressing assemblies to allow the active antimicrobial ingredient to percolate into the burned tissue. In order to not compromise the activity of the nanocrystalline silver from the silver dressing assemblies 112, pure water was used as a buffer. For the dressing assembly 212, it has been previously shown that pH 4.5 acetate buffer can be used to moisten the dressing assembly 212, without having the buffer have an antibacterial effect on P. aeruginosa in the short-term (i.e., within hours of application). Therefore, the dressing assemblies 212 adhering to the burns were then moistened daily with 100 uL of 50 mM sodium acetate buffer and the dressing assemblies 112 were moistened with MilliQ water, respectively.

E. Bioluminescence Imaging of the Mice

The low-light imaging system (Hamamatsu Photonics) consists of an intensified CCD camera mounted in a light-tight specimen chamber, fitted with a light-emitting diode, a set-up that allowed for a background gray-scale image of the entire mouse to be captured. In the photon-counting mode, an image of the emitted light from the bacteria was captured using an integration time of 2 min, at a maximum setting on the image-intensifier control module. By use of ARGUS software (Hamamatsu Photonics), the luminescence image was presented as a false-color image superimposed on top of the grayscale reference image. The image-processing component of the software calculated the total pixel values from the luminescence images of the infected wound area. The infection time was defined as the time during which any bioluminescence was present in the wound when measured at the most sensitive setting.

F. Monitoring of the Mice

During the experiment, mice underwent bioluminescence imaging immediately after adding bacteria and at 24 hourly intervals thereafter. Mice were also followed daily for weight and survival. When mice died, 5 mL sterile saline was injected into the abdominal cavity of mice, and then withdrawn and cultured on BHI agar plates to determine the presence of P. aeruginosa in the peritoneum of mice. Blood samples were also taken from the heart removed from dead mice and streaked on BHI agar plates.

G. Statistical Analysis

Survival curves were compared by the Kaplan-Meier log-rank test. P values <0.05 were considered statistically significant.

H. Results

The dressing assemblies 112 and 212 adhered extremely well to the surface of the burn when the assemblies had been previously moistened with acetate buffer or MilliQ water to render it flexible, as discussed above in section D. The adhesion time of dressing assemblies 112 and 212 was >16 days on all the mice that survived. The pieces of the dressing assemblies 112 and 212 were significantly bigger (>30 mm×30 mm, FIGS. 20(A), 20(B)) than the burn 94 itself (≈20 mm×10 mm, FIG. 20(C)) because the bacteria sometimes spread laterally into the skin beyond the burned area as observed by bioluminescence imaging.

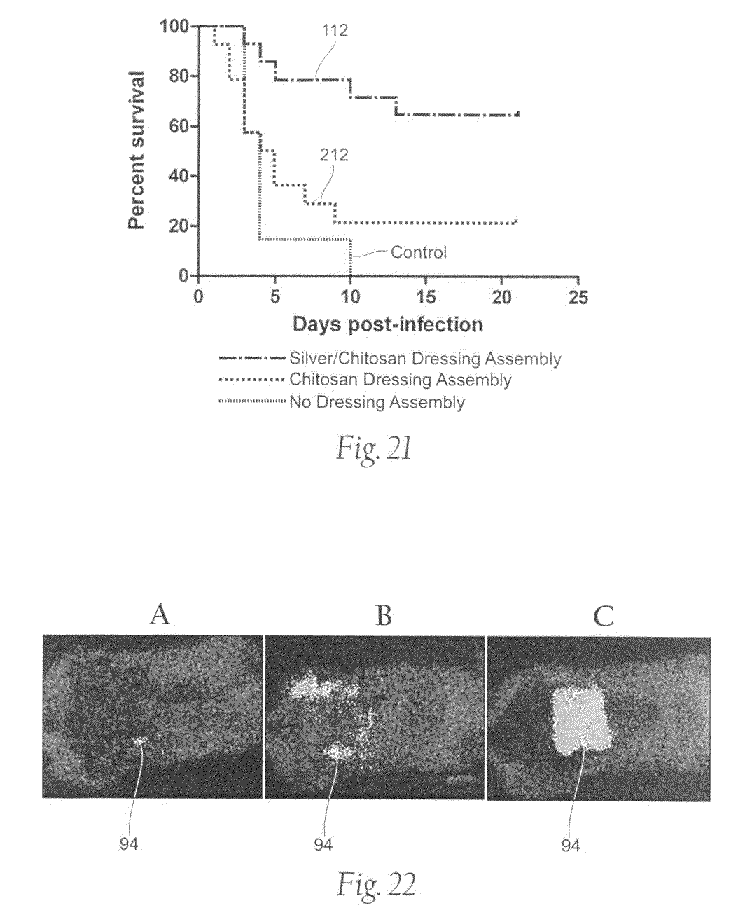

At 3 weeks post-infection, the survival rates of the mice 90 treated with the dressing assembly 112 (n=14), the mice 90 treated with the dressing assembly 212 (n=14), and the untreated mice 90 (n=7) were 64.3%, 21.4%, and 0%, respectively (FIG. 21). The survival curves were found to be significantly different between the mice 90 treated with the dressing assembly 112 and the mice 90 treated with the dressing assembly 212 (p=0.0082), and between the mice 90 treated with the dressing assembly 112 and the mice 90 that did not have any dressing assembly applied (p=0.0055). No significant difference was found between the survival curves of the mice 90 treated with the dressing assembly 212 and untreated mice 90 (p=0.68). In all three groups of the mice 90, most of the fatalities (15 out of 20) occurred between 2 to 5 days post-infection.

FIG. 22 shows the representative bioluminescence images (bit range=3) of mice with P. aeruginosa infected burns 94 with the dressing assembly 112 applied to the burn 94 (FIG. 22(A)), with the dressing assembly 212 applied to the burn 94 (FIG. 22(B)), and with no treatment on the burn 94 (FIG. 22(C)), at day 4 post-infection. As compared to the untreated mice, the dressing assembly 212 (FIG. 22(B)) appeared to slow the infection from spreading out of the burned area, while the dressing assembly 112 (FIG. 22(A)) appeared to significantly limit the area of infection.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A wound dressing comprising chitosan and silver nanoparticles.

2. A method for producing a wound dressing comprising
providing an aqueous mixture including a chitosan biomaterial and silver nanoparticles;
placing the aqueous mixture in a mold;
freezing the aqueous mixture within the mold by cooling the mold and aqueous mixture according to prescribed conditions to form a frozen chitosan and silver nanoparticle structure within the mold; and
removing water from the frozen chitosan and silver nanoparticle structure by a prescribed freeze-drying process to form a sponge-like chitosan and silver nanoparticle wound dressing saving a thickness and a density.

3. A method according to claim 2
further including compressing the sponge-like chitosan and silver nanoparticle wound dressing by the application of heat and pressure to reduce the thickness and increase the density of the sponge-like chitosan and silver nanoparticle wound dressing.

4. A method according to claim 2
further including preconditioning the chitosan and silver nanoparticle wound dressing by heating the chitosan and silver nanoparticle wound dressing according to prescribed conditions.

5. A method of treating a wound comprising
providing a wound dressing comprising chitosan and silver nanoparticles; and
applying the wound dressing to a wound site.

6. A wound dressing comprising chitosan and nanofibers.

7. A wound dressing according to claim 6
wherein the nanofibers comprise chitin.

8. A method of treating a wound comprising
providing a wound dressing comprising chitosan and nanofibers; and
applying the wound dressing to a wound site.

9. A method according to claim 8
wherein the nanofibers comprise chitin.

10. A method for producing a wound dressing comprising
forming chitosan in a nanofiber form,
re-acetylating the chitosan in nanofiber form to create chitin in nanofiber form,
providing an aqueous solution including a chitosan biomaterial and the chitin in nanofiber form;
placing the aqueous solution in a mold;
freezing the aqueous solution within the mold by cooling the mold and aqueous solution according to prescribed conditions to form a frozen chitosan and chitin nanofiber structure within the mold; and
removing water from the frozen chitosan and chitin nanofiber structure by a prescribed freeze-drying process to form a sponge-like chitosan and chitin nanofiber wound dressing.
